(19) United States
(12) Patent Application Publication
(10) Pub. No.: US 2022/0047183 A1
(43) Pub. Date: Feb. 17, 2022

(54) SELF-POWER SENSOR

(71) Applicant: INSPIRE MEDICAL SYSTEMS, INC., Golden Valley, MN (US)

(72) Inventors: Kevin Verzal, Lino Lakes, MN (US); Dave Dieken, Minneapolis, MN (US); John Rondoni, Plymouth, MN (US)

(73) Assignee: INSPIRE MEDICAL SYSTEMS, INC., Golden Valley, MN (US)

(21) Appl. No.: 17/458,715

(22) Filed: Aug. 27, 2021

Related U.S. Application Data
(63) Continuation of application No. 16/474,331, filed on Jun. 27, 2019, now abandoned.
(60) Provisional application No. 62/522,862, filed on Jun. 21, 2017.

(51) Int. Cl. A61B 5/113; A61B 5/08; A61B 5/00; H02K 35/02; H02N 1/08; H02N 2/18

(52) U.S. Cl. CPC A61B 5/113; A61B 5/08; A61B 5/686; A61B 2562/0219; H02N 1/08; H02N 2/186; A61B 2560/0214; H02K 35/02

(57) ABSTRACT

A self-powered sensor to produce an output signal corresponding to physiologic change and methods of use.

### CROSS-REFERENCE

This application is a continuation of U.S. application Ser. No. 16/474,331, filed Jun. 27, 2019, which is a 35 U.S.C. § 371 National Phase application that claims priority to International Application No. PCT/US18/38773, filed Jun. 21, 2018, which claims the benefit of the U.S. Provisional Patent Application No. 62/522,862, filed Jun. 21, 2017, the entire teachings of each of which are incorporated by reference herein.

### BACKGROUND

Sensors are widely employed to obtain physiologic information about a patient. Such sensors may be implantable within or external to a patient's body.

### BRIEF DESCRIPTION OF THE DRAWINGS

FIG. 1A is a block diagram schematically representing an example sensor.

FIG. 1B is a block diagram schematically representing an example receiving device.

FIG. 2 is a block diagram schematically representing an example device including an example mechanical-to-electric energy conversion element.

FIG. 3 is a block diagram schematically representing an example processing element.

FIG. 4A is a block diagram schematically representing an example energy conversion element.

FIG. 4B is a block diagram schematically representing an example piezoelectric element.

FIGS. 5-6 are each a diagram schematically representing an example energy conversion element.

FIGS. 7-8 are each a block diagram schematically representing an example sensor.

FIGS. 9A is a diagram schematically representing an example method of implanting and/or example implanted system within a patient's body.

FIG. 9B is a diagram schematically representing an example method of implanting a self-powered sensor and/or an example implantable system including a self-powered sensor.

FIG. 10 is a flow diagram schematically representing an example sensing method.

FIGS. 11-21 are each a block diagram schematically representing aspects of an example sensing method.

FIG. 22 is a flow diagram schematically representing an example sensing method.

FIG. 23 block diagram schematically representing aspects of an example sensing method.

FIG. 24 is a flow diagram schematically representing an example sensing method.

FIGS. 25-28 are each a block diagram schematically representing aspects of an example sensing method.

FIG. 29-32 are each a flow diagram schematically representing an example energy conversion method.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

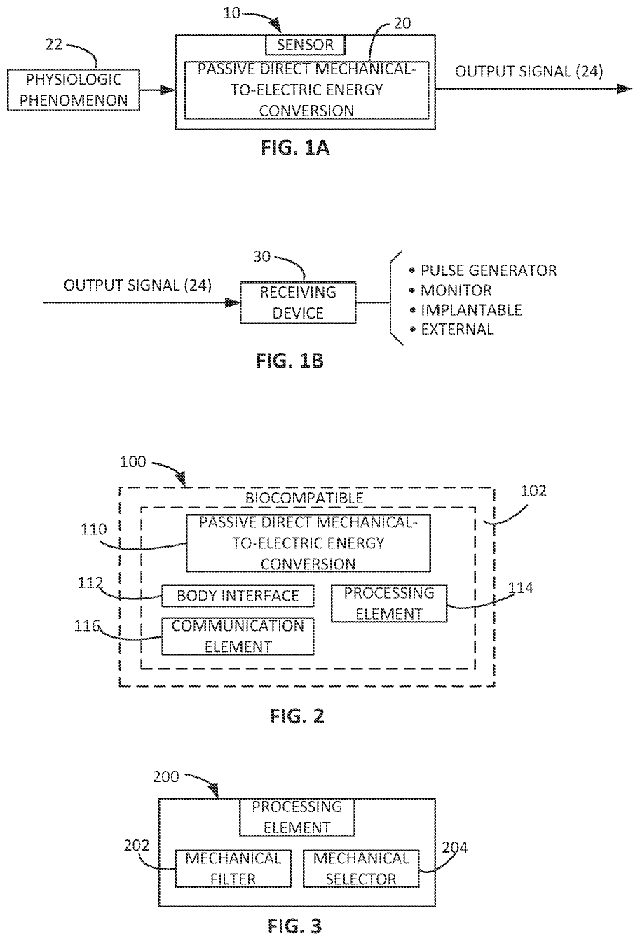

FIG. 1A is a block diagram of an example sensor 10. In at least some examples, the sensor 10 may create a bioelectric signal that can be directly sensed (e.g. captured) without the need for input power. In some instances, the sensor may sometimes be referred to as a zero-external-input power sensor. In some instances, the sensor may sometimes be referred to as a bioelectric prosthetic to the extent that at least some elements of the device are attachable (or otherwise couplable) to a portion of the body at which a physiologic phenomenon occurs which is to be measured, sensed, etc. Via such attachment or fixation, the device may move in unison with the body portion (or absorb body movement) and as such functions with the body in producing a bioelectric signal from mechanical behavior in the region at which the sensor is placed. Via such arrangements, the device may sometimes be referred to as and/or comprise a mechanical-to-electrical energy conversion element 20. In some examples, this conversion element 20 may be referred to as being passive in that its operation does not depend on first sending a signal in order to sense information (such as in SONAR, DOPPLER) and/or that the conversion element does not require input power from an external source in order to capture the desired physiologic phenomenon 22.

In some instances, it may be said that the conversion element 20 is passive to the extent that its behavior in producing an output signal 24 is solely a response to a physiologic phenomenon. Stated differently, in some examples the output signal 24 may be produced in a single step of capture and conversion of the mechanical physiologic phenomenon.

The output signal 24 may be received by a receiving element 30, as shown in FIG. 1B. In some examples, the receiving element 30 may comprise a device which is implantable or external to the patient's body. In some examples, the receiving element 30 may comprise a pulse generator (PG), which may be implantable and/or external to the patient's body. In some examples, instead of an IPG, the receiving element 30 comprises a monitor to observe and evaluate physiologic phenomenon 22.

With further reference to FIG. 1A, in some instances, the example conversion element 20 may sometimes be referred to as a direct mechanical-to-electrical conversion element to the extent that the output signal 24 may directly flow from the element mimicking the mechanical behavior (e.g. physiologic phenomenon 22) of the body portion to which the conversion element 20 is attached.

The mechanical behavior may be a movement of muscles, bones, soft tissue and/or a movement of a liquid or gas through a lumen, vasculature, etc. of the body. The movement may be a large scale movement (e.g. walking) and/or may be a smaller scale movement (e.g. respiration, vibration, etc.). In some instances, the movement takes the form of wave or other energy propagating through the portion of the body at which the example sensor is located. In some examples, the physiologic phenomenon 22 may be thermal, chemical, etc.

Some such arrangements may be understood as allowing extremely low power or no-power sensing of any biomechanical signal upon physically locating the sensor on or in the body at the behavior to be sensed. In some instances, some such arrangements also may be understood according to an action mechanism (e.g. conversion element) which is both a power source and a mechanism which captures the data of interest. Stated differently, the action mechanism enables real-time powering without any transfer/consumption of power from other sources (in or outside of the sensor), and therefore at least some such example sensors may omit a storage element. Several example conversion elements are described below.

In some examples, the sensor 10 may be tuned (e.g. sized) to provide at least some of the converted energy as power to an end point. In some such examples, providing power (e.g. via output signal 24) to an end point may be implemented via a conductor (e.g. wire) extending between the sensor and the end point.

In some examples, the output signal(s) 24 produced via such example implementations of sensor 10 may be filtered and undergo signal extraction before decisions can be made based on them. In some examples, the power used to process the output signal 24 and/or for other purposes may be derived from the mechanical signal captured/produced via this sensor 10.

By providing an example sensor 10 which may operate without externally supplied power, the power demands on an associated implantable pulse generator (IPG), monitoring circuitry, etc. may be reduced, which in turn may allow smaller power sources (e.g. battery), less frequent recharging of rechargeable power sources, greater longevity of implanted devices (IPG) etc. Moreover, some such example sensors may be significantly smaller, thereby easing their implantation, enable insertion into smaller physiologic spaces, etc. In some examples, such arrangements may enable the use of a greater number of sensors than otherwise might be supportable by the power available from a pulse generator, monitoring circuitry, etc. In addition, some such example sensors may eliminate the infrastructure, control, and activity involved in transferring power from another implanted device (e.g. IPG) to the sensor.

Moreover, at least some example sensors may eliminate the use/presence of electrical power used in traditional sensors, which are typically powered via a battery source in a device (e.g. IPG) external to the sensor.

To the extent that energy may be harnessed via converting mechanical energy to electrical energy via such example sensors (e.g. 10 in FIG. 1A), it will be understood that in at least some examples, none of the energy captured via such conversion is transferred, stored, etc. for use in powering a device, component, etc. other than (e.g. external to) the sensor through which the power was captured, generated, etc.

While such an example sensor 10 may be used to sense a wide variety of physiologic phenomenon 22, in some examples sensor 10 may be used to sense respiratory information. In some examples, such sensed respiratory information may be used to monitor, detect, evaluate, diagnose, and/or treat sleep disordered breathing (SDB) such as hypopneas, obstructive sleep apnea (OSA), etc. Other sensed information may comprise detection and/or evaluation of apnea events, blood oxygenation, posture, motion, sleep quality, cardiac health, etc.

In some examples, sensor 10 may be implanted subcutaneously or percutaneously, or even transvenously (e.g. intravascularly). In some examples, whether transvenous or not, the sensor 10 may be implanted in a non-cardiac location of the body.

These examples, and further examples are described in association with at least FIGS. 2-32.

FIG. 2 is a block diagram schematically representing an example device 100. In some examples, the device 100 comprises at least some of substantially the same features and attributes as the sensor 20 in FIG. 1A, and in some examples, the device 100 may comprise one example implementation of the sensor 10 in FIG. 1A.

As shown in FIG. 2, in some examples device 100 may comprise a conversion element 110 (like conversion element 20 in FIG. 1A) which converts a mechanical signal/behavior associated with physiologic phenomenon into an electrical signal of power significant enough for robust processing, further use in physiologic monitoring, diagnosis, therapy, etc.

In some examples, device 100 may comprise a biocompatible barrier 102, such as a housing and/or coating. The housing may be a singular housing. In some such examples, at least some portions of a housing associated with the device 100 may be highly flexible and/or have a wide range of sizes/shapes to adapt to a wide variety of anatomical and physiologic environments. In some examples, such a highly flexible housing portions may be resilient and/or have shape memory behavior. Some such arrangements may enhance the ability of the housing (or housing portions) to wrap about and/or conform to particular anatomical and/or physiologic structures. In some examples, the housing may have at least some portions which are rigid and/or semi-rigid to adapt to particular anatomical and/or physiologic environments.

In some examples, the device 100 may comprise a communication element 116, such an electrical conductor (e.g. wires) to communicate the signal to other components, such as monitor. In some examples, the communication element 116 may comprise a wireless communication mechanism.

In some examples, the device 100 may comprise a body interface 112 to couple at least some portions of the device 100 relative to the body and in particular to couple at least the conversion element 110 to a body portion through the physiologic phenomenon of interest may be engaged, received, etc. via the conversion element 110. In some examples, the body interface 112 may comprise at least part of the housing/barrier 102.

In some examples, the device 100 may comprise signal processing element(s) 114 as desired. In some such examples, the processing element 114 may comprise the processing element 200 described below in association with at least FIG. 3.

FIG. 3 is a block diagram schematically representing an example processing element 200 forming part of or associated with a sensor 10 (FIG. 1A). As shown in FIG. 3, the processing element 200 may comprise a mechanical filter 202 and/or a mechanical selector 204. The mechanical filter 202 may remove certain frequencies from the electrical signal generated via the conversion element 20 and/or the mechanical selector 204 may select or be tuned to certain frequencies of interest, either (filtering or selecting) of which may desirably condition the output signal 24 prior to final processing by a remote monitor/processing circuitry. At least some of these arrangements may reduce overhead, such as via mechanical dampeners and/or focusing on essential frequencies/harmonics.

In some examples, a size and/or shape of biomechanical interface (e.g. body interface 112 in FIG. 2) may be selected to have a natural frequency related to the biological signals of interest (e.g. physiologic phenomenon 22). In some examples, a housing for the biomechanical interface (e.g. body interface 112 in FIG. 2) may be designed to be actuated solely by forces of a particular magnitude and/or frequency range of interest by selection of material stiffness, thickness, and size. Via at least some such example arrangements, electronic filtering circuits and their related power consumption may be minimized or eliminated.

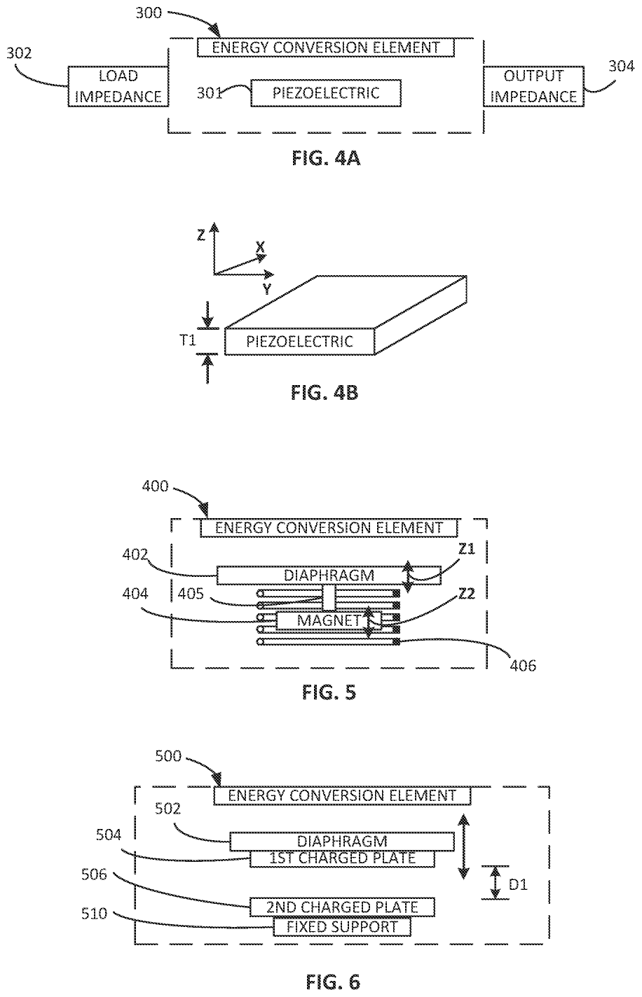

FIG. 4A is a side plan view schematically representing an example energy conversion element 300 comprising a piezoelectric element 301. In some examples, the conversion element 300 comprises one example implementation of the conversion element 20, 110 in FIGS. 1A-2 or conversion element 610 in FIG. 7.

In one aspect, the dashed lines 300 in FIG. 4A represent that the conversion element 300 may be embodied in a wide variety of housings and/or physical configurations, and is not necessarily packaged in a traditional “can.” In some examples, such housings may comprise at least some of substantially the same features and attributes as previously described in association with at least device 100.

As shown in FIG. 4A, the conversion element 300 may be associated with a load impedance 302 and an output impedance 304.

In some examples, piezoelectric element 301 may be selected appropriate for the load to be driven (internal electronics, transmitter, or just a signal up a lead wire). In some examples, the piezoelectric element 301 may comprise a single crystal piezoelectric element, which in some instances may enable driving more charge than polycrystalline devices. However, polycrystalline piezoelectric elements may be employed in some instances. Moreover, in some examples, the piezoelectric element 301 may comprise a shape adapted to increase charge generation given a particular movement profile. In some examples, multiple crystals can be combined to improve output.

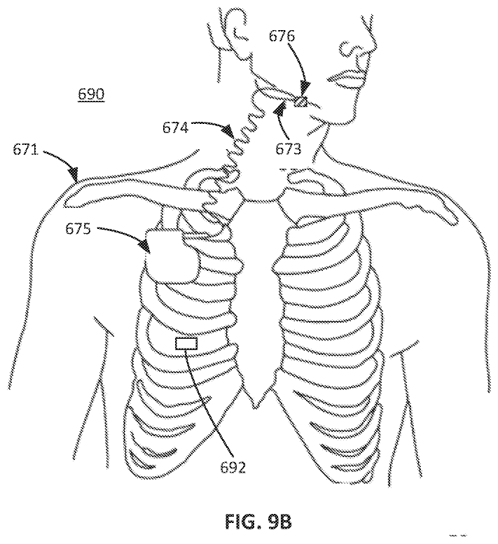

In some examples, the piezoelectric element 301 may comprise a minimal housing, thereby simplifying the design and reducing costs while maximizing flexibility of the piezoelectric element. In some such examples, the piezoelectric element 301 may comprise a hermetic coating/encapsulation with materials like a liquid crystal polymer (LCP), such as but not limited to a polyimide material. In some examples, a dielectric coating (e.g. parylene) can be combined with other coating materials and/or layers to create a multi-layered hermetic or adequately near hermetic housing. In some examples, such encapsulated piezoelectric element 301 may be connected to lead wires and run directly into the IPG. In some examples, such a piezoelectric element 301 acting as a conversion element 300 may be embedded into an IPG (e.g. medical device 675 in FIGS. 9A, 9B).

In some examples the example conversion element 300 may comprise a relatively low or moderate output impedance 304 (e.g. increased capacitance) which is substantially less than an output impedance of a traditional piezoelectric element. In some such examples, the relatively low/moderate output impedance 304 may be implemented via utilizing relative large x, y dimensions and/or a smaller z dimension (e.g. thickness T1), as shown in FIG. 4B. Alternatively, such lower output impedances may be implemented via material properties, such as a chemical composition or crystalline structure.

At least some such example arrangements of piezoelectric element 301 having low output impedance 304 may facilitate the output signal 24 (FIG. 1A) being of sufficient magnitude to enable robust signal processing of the output signal 24 by a device or component receiving the output signal 24.

Such example arrangements stand in sharp contrast to some traditional piezoelectric arrangements which exhibit a large output impedance, such that a load impedance may reduce (e.g. attenuate) the output signal of the traditional piezoelectric to a sufficiently low level to undesirably and significantly hinder the strength and/or quality of the output signal received at the processing circuitry (which may be external/remote to the sensor and which may be a piezoelectric element). In some instances, the load impedance can be due to the nature of electronics, or parasitics caused by the particular implantable application (e.g. capacitance from sensor and leads to tissue, resistance from sensor and/or leads to tissue where insulation is not ideal, design/length of conductor/wire from the sensor to the external circuitry, etc.). Because of these issues, at least some traditional sensors including a piezoelectric element may incorporate or be associated with processing circuitry (e.g. one or more of MOSFET, microcontroller, ASIC, passive components, etc.) as part of the sensor of in order to buffer the output signal, such as via reducing the output impedance.

However, at least some example piezoelectric elements 301 (FIG. 4A) omit such internal processing circuitry (e.g. e.g. one or more of MOSFET, microcontroller, ASIC, passive components, etc.), which in turn eliminates a power demand (e.g. requirement for input power from an external source). In some such example arrangements in which the piezoelectric element 301 of the present disclosure omits such internal processing circuitry, a load impedance 302 may be increased via decreasing a load capacitance and/or increasing a load resistance. For instance, some example piezoelectric elements 301 may incorporate a thicker insulator over the piezoelectric element 301 and/or associated lead, may reduce an area of conductive elements (e.g. wire) associated with conversion element 300/piezoelectric element 301, and/or may reduce an input capacitance of the remote/external processing circuitry via using electronics MOSFET source follower (or one or more of a microcontroller, ASIC, passive components, etc.). In other instances such as when the insulation of one of the conductors is not ideal, a decreased load resistance in one of the two signal conductors can result, which may be mitigated by using a differential amplifier/buffer circuit at the external/remote processing circuitry.

In some examples, the piezoelectric element 301 may be replaced with other modalities such as triboelectric, pyroelectric, etc.

FIG. 5 is a side plan view schematically representing an example energy conversion element 400. In some examples, the example energy conversion element 400 comprises one example implementation of the conversion element 20, 110 in FIGS. 1A-2 or conversion element 610 in FIG. 7. In general terms, the energy conversion element 400 comprises an electromagnetic sensing element. As shown in FIG. 5, the conversion element 400 comprises a diaphragm 402, a magnet 404, and an electrically conductive coil 406. In one aspect, the dashed lines 400 represent that the conversion element 400 may be embodied in a wide variety of housings and/or physical configurations, and is not necessarily packaged in a traditional “can.” In some examples, such housings may comprise at least some of substantially the same features and attributes as previously described in association with at least device 100.

With further reference to FIG. 5, the magnet 404 is coupled relative to the diaphragm 402 via an element 405. With coil 406 in a fixed position, movement of the diaphragm in response to physiologic phenomenon causes movement of the magnet 404 relative to the coil 406, thereby generating a voltage at coil 406 via electromagnetic principles. In some examples, the coil 406 may comprise a relatively low output impedance, which in some instances, may reduce or eliminate adaptations that might otherwise be included when a sensing element has a relatively high output impedance. In some examples, the diaphragm 402 may comprise at least a flexible, resilient portion or material.

FIG. 6 is a side plan view schematically representing an example energy conversion element 500. In some examples, the example energy conversion element 500 comprises one example implementation of the conversion element 20, 110 in FIGS. 1A-2 or conversion element 610 in FIG. 7. In general terms, the energy conversion element 500 may comprise an electret capacitor.

As shown in FIG. 6, the conversion element 500 comprises a diaphragm 502, first and second charged plates 504, 506, and a fixed support 510. In one aspect, the dashed lines 500 represent that the conversion element 500 may be embodied in a wide variety of housings and/or physical configurations, and is not necessarily packaged in a traditional “can.” In some examples, such housings may comprise at least some of substantially the same features and attributes as previously described in association with at least device 100.

With further reference to FIG. 6, the first charged plate 504 is spaced apart from the second charged plate 506 by a distance D1, with first charged plate 504 being coupled relative to the diaphragm 502. One or both of plates 504, 506 have a permanent charge deposited on them. With second plate 506 in a fixed position (e.g. per fixed support 510), movement of the diaphragm 502 in response to a physiologic phenomenon causes movement of the first charged plate 504 relative to the second plate 506, thereby generating a voltage as an output signal. In some examples, the coil 406 may comprise a relatively low output impedance (e.g. increased capacitance), which in some instances, may reduce or eliminate adaptations that might otherwise be included when a sensing element has a relatively high output impedance. In some examples, the diaphragm 502 may comprise at least a flexible, resilient portion or material.

FIG. 7 is a block diagram schematically representing an example sensor 600. In some examples, the example sensor 600 may comprise at least some of substantially the same features and attributes as the example sensor as previously described in association with FIGS. 1A-6, except further comprising a storage element 630 and/or an energy harvesting element 620.

In some examples, the sensor 600 may comprise a mechanical-to-electrical conversion element 610 like the conversion elements 300, 400, 500 as previously described in association with at least FIGS. 4A-6. In some examples, the sensor 600 may have a housing comprising at least some of substantially the same features and attributes as at least some of the previously described examples.

In some examples, the energy harvesting element 620 may comprise an element separate from the conversion element 610 while in some examples, the energy harvesting element 620 and conversion element 610 may be embodied in a single structure or monolithic structure.

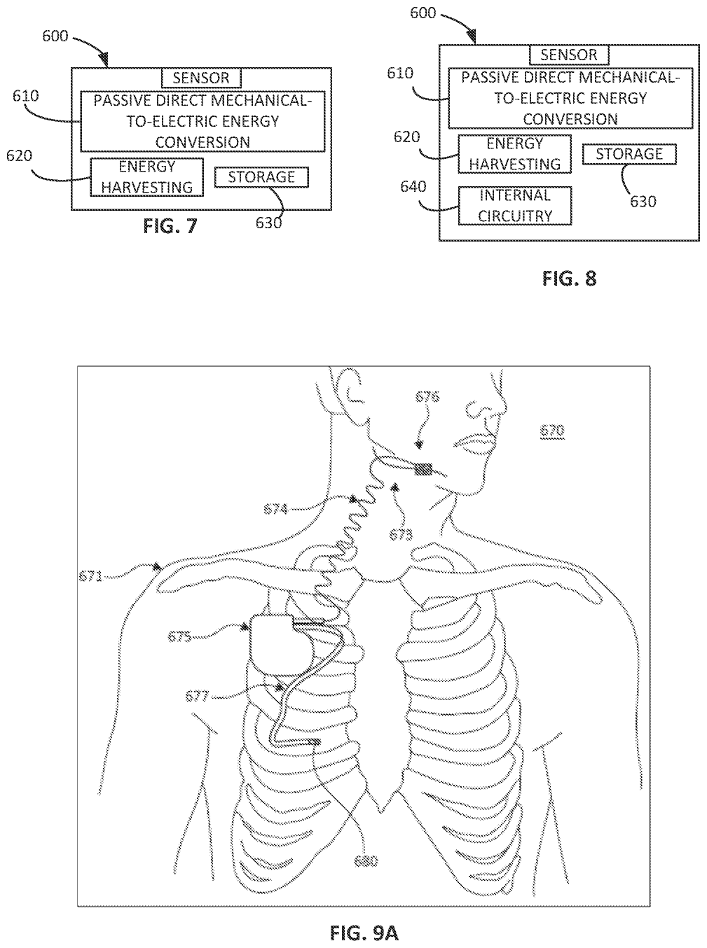

In some examples, the energy harvesting element 620 may comprise a piezoelectric element or MEMS electret capacitor. In some such examples, a motion of the body and/or an externally applied vibration may result in the harvested energy. The energy harvesting element may produce a voltage which can be rectified via diodes and stored in storage element 630 (e.g. a capacitor). In some such examples, the sensor 600 may comprise its own/internal circuitry 640 for processing, amplification, and/or other purposes, as shown in FIG. 8 with such circuitry being powered via the storage element 630.

In some examples, the mechanical-to-electric conversion element 610 may comprise a piezoelectric element to sense motion, pressure, and/or strain. In some such examples, the conversion element 610 also may serve as the energy harvesting element 620.

In some examples, the conversion element 610 may comprises a motion sensing element implemented via an accelerometer.

In some examples, the conversion element 610 may comprise a motion sensing element implemented via a capacitor. In some examples, this conversion element may comprise at least some of substantially the same features and attributes as the conversion element in FIG. 6 by which a capacitance change can be measured.

In some examples, the sensor 600 may comprise and/or be associated with an impedance sensing pair in which a current is sent through tissue via two or more electrodes and a resulting voltage measured.

In some examples, the sensor 600 may comprise and/or be associated with a voltage measured across two or more electrodes, such as via modalities like an ECG, EEG, EMG, etc.

In some examples, the signal transmission from the sensor 600 (or electronics/processing circuitry associated with the sensor 600) may be wireless or wired (e.g. an implanted lead).

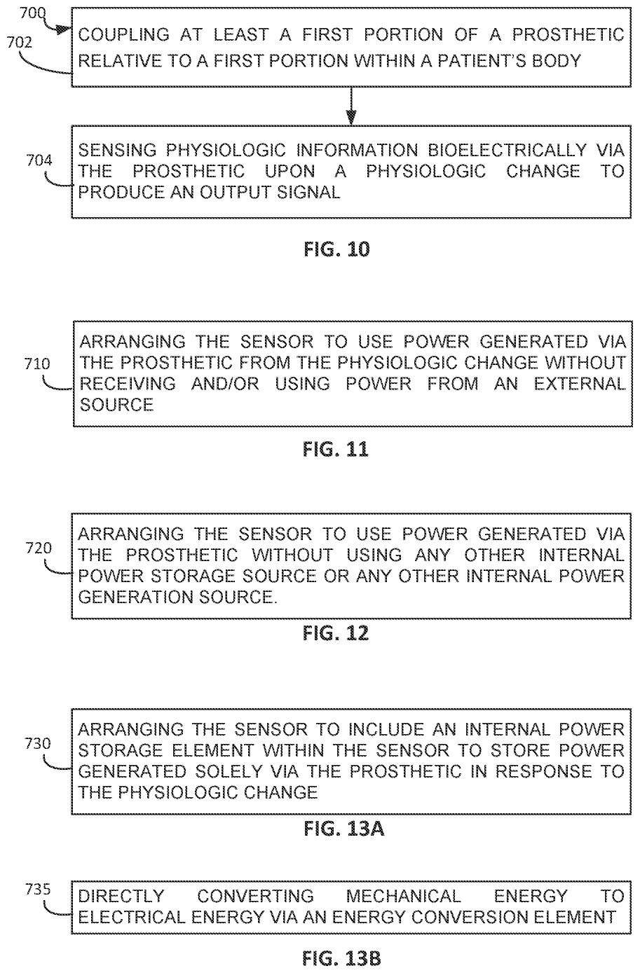

FIG. 9A is a diagram schematically representing an example method 670 of implanting a medical device. In some examples, method 670 comprises one example implementation of at least some of the various examples described in association with at least FIGS. 1A-8 and/or FIGS. 10-32.

As illustrated in FIG. 9A, in some examples method 670 comprises surgically positioning medical device 675 within a patient's body 671. In some such examples, medical device 675 is implanted within a pectoral region, although medical device 675 may be implanted elsewhere within the body 671. In some examples, a stimulation lead 674 and/or sensor lead 677 also may be implanted within body 671 in which subcutaneously tunneling is typically performed to place the respective leads in their desired positions within the body 671. After such tunneling, the respective leads 674, 677 may be connected electrically and/or mechanically to the medical device 675.

In some examples, medical device 675 may comprise an electronic medical device, such as but not limited to, an implantable pulse generator (IPG) for at least performing sleep apnea monitoring, therapy, diagnosis, among other physiologic-related functions. In some examples, medical device 675 may comprise additional or other structures, and perform additional or other functions. In some examples, medical device 675 may comprise a monitoring device which does not provide neurostimulation but which monitors physiologic parameters and/or other information.

In some examples, the stimulation lead 674 includes a stimulation element 676 (e.g. electrode portion, such a cuff electrode) and extends from the medical device 675 so that the stimulation element 676 is positioned in contact with a desired nerve 673 to stimulate nerve 673 for restoring upper airway patency. In some examples, the desired nerve comprises a hypoglossal nerve.

In some examples, device 675 comprises includes at least one sensor portion 680 (electrically and mechanically coupled to the medical device via lead 677) positioned in the patient's body 671 for sensing physiologic conditions, such as but not limited to, respiratory effort.

In some examples, the sensor portion 680 detects respiratory effort including respiratory patterns (e.g., inspiration, expiration, respiratory pause, etc.). In some examples, this respiratory information is employed to trigger activation of stimulation element 676 to stimulate a target nerve 673. Accordingly, in some examples, the IPG 675 receives sensor waveforms (e.g. one form of respiratory information) from the respiratory sensor portion 680, thereby enabling the IPG 675 to deliver electrical stimulation according to a therapeutic treatment regimen in accordance with examples of the present disclosure. In some examples, this respiratory information can be used to collect diagnostics on device effectiveness.

In some examples, sensor portion 680 comprises at least some of substantially the same features and attributes described in association with the examples of at least FIGS. 1A-8 and/or FIGS. 9B-32. Accordingly, sensor portion 680 may comprise a self-powered sensor including a passive, direct mechanical-to-electrical energy conversion element (e.g. 20, 110, 300, 400, 500, 610, etc.) and/or associated processing, storage, energy harvesting elements, etc. Therefore, despite the presence of lead 677, the sensor portion 680 does not receive power from medical device 675.

In some examples, the sensing and stimulation system for treating sleep disordered breathing (such as but not limited to obstructive sleep apnea) is a totally implantable system which provides therapeutic solutions for patients diagnosed with obstructive sleep apnea. In other examples, one or more components of the system are not implanted in a body of the patient. Whether partially implantable or totally implantable, in some examples the system is designed to stimulate an upper-airway-patency-related nerve during some portion of the repeating respiratory cycle to thereby prevent obstructions or occlusions in the upper airway during sleep.

FIG. 9B is a diagram schematically representing an example method 690 having substantially the same features and attributes as method 675, except omitting lead 677 and including a lead-less sensor 692. In other words, in some examples method 690 comprises implanting sensor 692 without tunneling a path between a location of the medical device 675 and a location of the sensor 692, thereby providing a less invasive implant procedure. Accordingly, instead of being connected and/or communicating via lead 677, the sensor 692 may comprise a wireless communication element (e.g. 116 in FIG. 2) to communicate with the medical device 675 or an external medical device. In addition, because sensor 692 comprises substantially the same features and attributes as sensor 680 such wireless communication element is not used to receive power from (or to transmit power to) the medical device. Instead, sensor 692 is self-powered in the same manner as the example sensor 680.

FIG. 10 is a flow diagram schematically representing an example sensing method 700. In some examples, method 700 may be performed via at least some of the sensors, energy conversion elements, devices, methods, etc. as described in association with the examples of at least FIGS. 1A-9B, 11-32. In some examples, method 700 may be performed via at least some of the sensors, energy conversion elements, devices, methods, etc. other than those previously described in association with the examples of at least FIGS. 1A-9B, 11-32.

As shown in at 702 in FIG. 10, method 700 may comprise coupling at least a first portion of a prosthetic relative to a first portion within a patient's body. At 704, method 700 may comprise sensing physiologic information bioelectrically via the prosthetic upon a physiologic change to produce an output signal. For instance, the output signal may comprise a voltage which corresponds to and/or is representative of the physiologic change occurring, which in turn may be further representative in some examples of a physiologic phenomenon driving the physiologic change.

As shown at 710 in FIG. 11, in some examples method 700 may further comprise arranging the sensor to use power generated via the prosthetic from the physiologic change without receiving and/or without using power from an external source, such as a medical device (e.g. 675 in FIGS. 9A, 9B) in some examples.

As shown at 720 in FIG. 12, in some examples method 700 may further comprise arranging the sensor to use power generated via the prosthetic without using any other internal power storage source and/or any other internal power generation source.

As shown at 730 in FIG. 13A, in some examples method 700 may further comprise arranging the sensor to include an internal power storage element within the sensor to store power generated solely via the prosthetic in response to the physiologic change.

As shown at 735 in FIG. 13B, in some examples sensing physiologic information in method 700 may comprise directly converting mechanical energy to electrical energy via an energy conversion element, which in some examples may comprise a piezoelectric element and/or other mechanical-to-electrical energy conversion element(s).

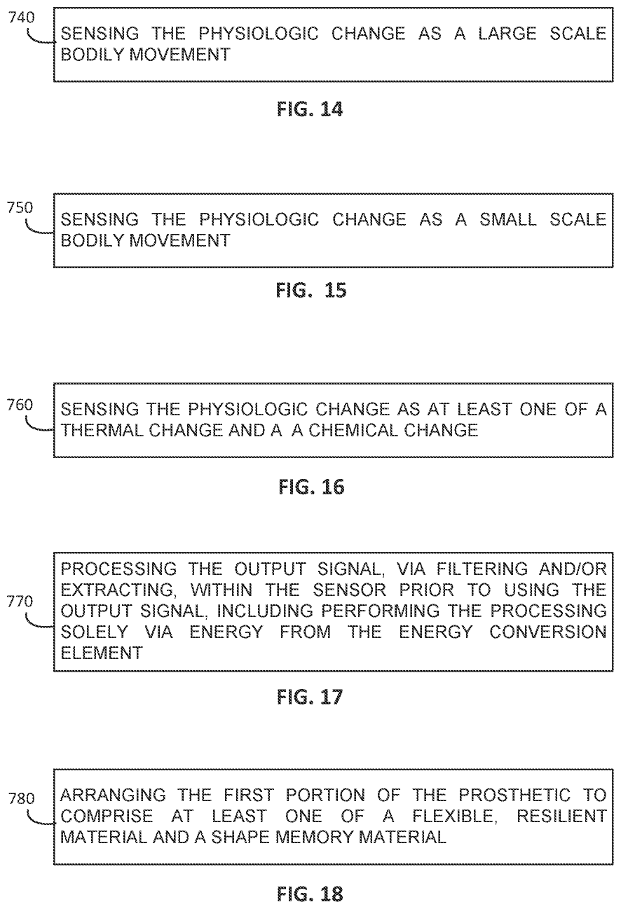

As shown at 740 in FIG. 14, in some examples method 700 may further comprise sensing the physiologic change as a large scale bodily movement. In some examples, a large scale bodily movement may comprise locomotion of the body or movement of a limb, sitting, standing, etc.

As shown at 750 in FIG. 15, in some examples method 700 may further comprise sensing the physiologic change as a small scale bodily movement, such as a vibration, percussion, acoustic sounds, etc.

In some examples, the small scale bodily movement may comprise motion, pressure, strain, etc. associated with movement(s) of portions of the body involved in respiration. In some such examples, these respiratory small scale bodily movements may comprise apnea events (e.g. obstructive, hypopnea, etc.) or regular respiratory cycles.

As shown at 760 in FIG. 16, in some examples method 700 may further comprise sensing the physiologic change as at least one of a thermal change or a chemical change. In some such examples, an energy conversion element (e.g. 20, 110, 300, 400, 500, 610) may comprise a direct thermal-to-electric energy conversion element and/or a direct chemical-to-electric energy conversion element instead of a mechanical-to-electric energy conversion element.

As shown at 770 in FIG. 17, in some examples method 700 may further comprise processing the output signal, via filtering and/or extracting, within the sensor prior to using the output signal with this processing including performing the processing solely via energy from the energy conversion element. In some examples, the filtering may comprise a mechanical filtering (e.g. 202 in FIG. 3) while in some examples the extracting may comprise a mechanical extracting (e.g. 204 in FIG. 3). In some examples, the mechanical extracting may sometimes be referred to as mechanical selecting and/or implemented as mechanical selecting.

As shown at 780 in FIG. 18, in some examples method 700 may further comprise arranging the first portion of the prosthetic to comprise at least one of a flexible resilient material and a shape memory material.

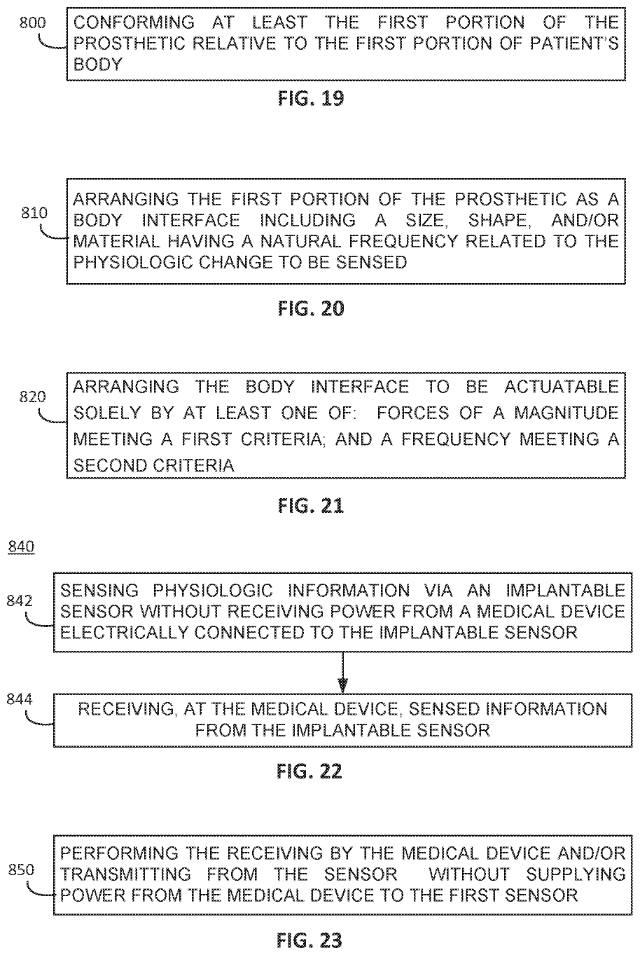

As shown at 800 in FIG. 19, in some examples method 700 may further comprise conforming at least the first portion of the prosthetic relative to the first portion of the patient's body.

As shown at 810 in FIG. 20, in some examples method 700 may further comprise arranging the first portion of the prosthetic as a body interface including a size, shape, and/or material having a natural frequency related to the physiologic change to be sensed, such as (but not limited to) previously described in association with at least FIG. 2. It will be understood that, in at least some examples, the natural frequency may comprise a range of frequencies related to (e.g. corresponding to) a frequency or range of frequencies associated with the physiologic change to be sensed.

As shown at 820 in FIG. 21, in some examples method 700 may further comprise arranging the body interface to be actuatable solely by at least one of forces of a magnitude meeting a first criteria or a frequency meeting a second criteria.

FIG. 22 is a flow diagram schematically representing an example sensing method 840. In some examples, method 840 may be performed via at least some of the sensors, energy conversion elements, devices, methods, etc. as previously described in association with the examples of at least FIGS. 1A-21, 23-32. In some examples, method 840 may be performed via at least some of the sensors, energy conversion elements, devices, methods, etc. other than those previously described in association with the examples of at least FIGS. 1A-21, 23-32.

As shown at 842 in FIG. 22, in some examples method 840 may comprise sensing physiologic information via an implantable sensor without receiving power from a medical device electrically connected to the implantable sensor. At shown at 844, method 840 may comprise receiving, at the medical device, sensed physiologic information from the implantable sensor.

As shown at 850 in FIG. 23, in some examples method 850 may comprise performing the receiving by the medical device, and/or transmitting the information from the sensor, without supplying power from the medical device to the implantable sensor.

In some such examples, the medical device may comprise an implantable medical device while in some such examples, the medical device may be external to the patient's body. In either case, in some examples, the information is wireless communicated from the sensor (e.g. transmitted from) to the medical device (e.g. received by).

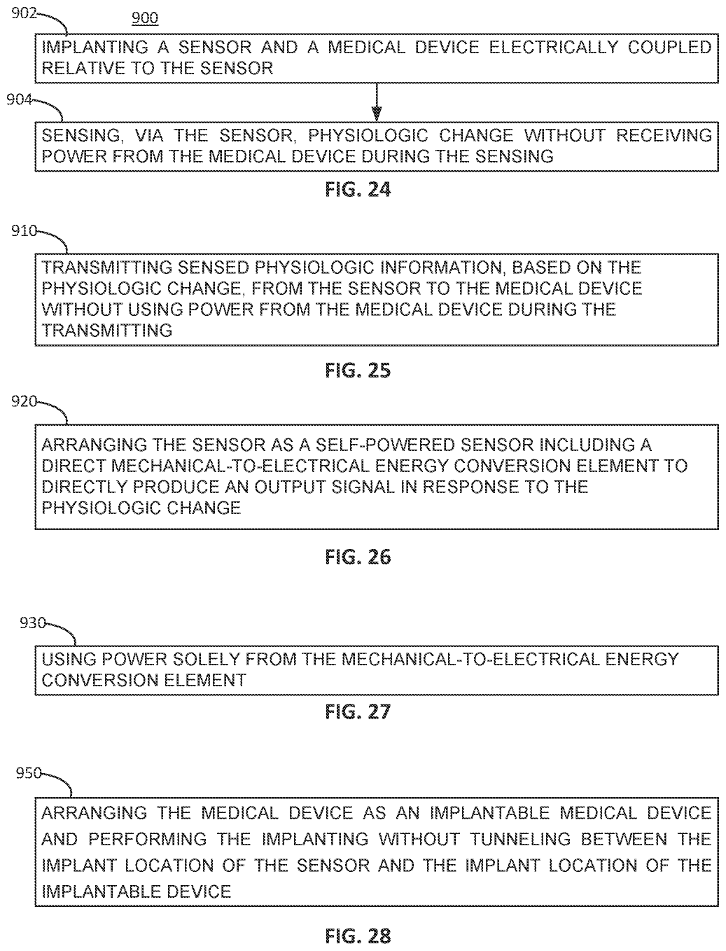

FIG. 24 is a flow diagram schematically representing an example sensing method 900. In some examples, method 902 may be performed via at least some of the sensors, energy conversion elements, devices, methods, etc. as described in association with the examples of at least FIGS. 1A-23, 25-32. In some examples, method 900 may be performed via at least some of the sensors, energy conversion elements, devices, methods, etc. other than those described in association with the examples of at least FIGS. 1A-23, 25-32.

As shown at 902 in FIG. 24, in some examples method 900 may comprise implanting a sensor and a medical device electrically coupled relative to the sensor. As shown at 904, method 900 may comprise sensing, via the sensor, physiologic change without receiving power from the medical device during the sensing.

As shown at 910 in FIG. 25, method 900 may further comprise transmitting sensed physiologic information, based on the physiologic change, from the sensor to the medical device without using power from the medical device during the transmitting.

As shown at 920 in FIG. 26, method 900 may further comprise arranging the sensor as a self-powered sensor including a direct mechanical-to-electrical energy conversion element to directly produce an output signal in response to the physiologic change.

As shown at 930 in FIG. 27, method 900 may further comprise using power solely from the direct mechanical-to-electrical energy conversion element.

As shown at 950 in FIG. 28, method 900 may further comprise arranging the medical device as an implantable medical device and performing the implanting without tunneling between the implant location of the sensor and the implant location of the implantable device.

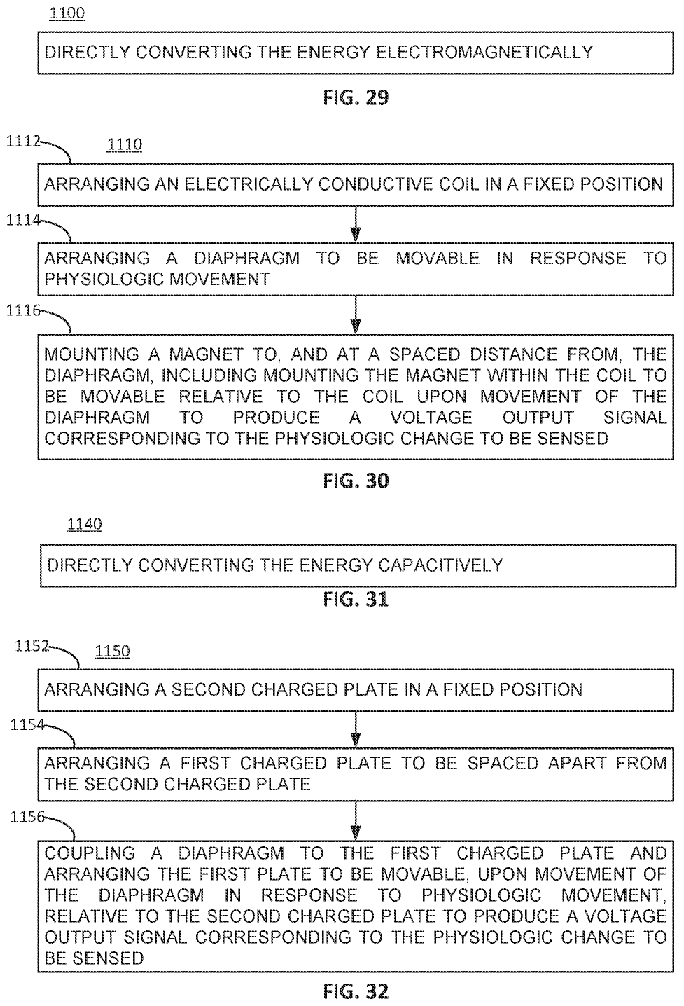

FIG. 29 is a diagram schematically representing an example method 1100 of directly converting the energy electromagnetically. As shown at 1110 in FIG. 30, in some examples method 1100 may comprise arranging an electrically conductive coil in a fixed position (1112) and arranging a diaphragm to be movable in response to physiologic movement (1114). As further shown at 1116 in FIG. 30, in some examples method 1100 may comprise mounting a magnet to, and at a spaced distance from, the diaphragm, including mounting the magnet within the coil to be movable relative to the coil upon movement of the diaphragm to produce a voltage output signal corresponding to the physiologic change to be sensed. In some examples, the method 1100 may be implemented via at least some of the elements of the energy conversion element described in association with at least FIG. 5. More broadly speaking, methods 1100, 1110 in FIGS. 29-30 may be implemented in association with, or as part of, any one of the examples described in association with FIGS. 1A-28.

FIG. 31 is a diagram schematically representing an example method 1140 of directly converting the energy capacitively. As shown at 1150 in FIG. 32, in some examples method 1140 may comprise arranging a second charged plate in a fixed position (1152) and arranging a first charged plate to be spaced apart from the second charged plate (1154). As further shown at 1156, method 1140 may further comprise coupling a diaphragm to the first charged plate and arranging the first charged plate to be movable, upon movement of the diaphragm in response to physiologic movement, relative to the second charged plate to produce a voltage output signal corresponding to the physiologic change to be sensed. In some examples, the method 1140 may be implemented via at least some of the elements of the energy conversion element described in association with at least FIG. 6. More broadly speaking, methods 1140, 1150 in FIGS. 31-32 may be implemented in association with, or as part of, any one of the examples described in association with FIGS. 1A-28.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. (canceled)

2. A method comprising:
locating a self-powered physiological sensor within a patient's body in contact with native tissue that moves in response to respiratory effort;
sensing respiratory effort information of the patient via the located self-powered physiological sensor without receiving or using power from an external source; and
delivering a signal indicative of the respiratory effort information from the self-powered physiological sensor to a separate device.

3. The method of claim 2, wherein the self-powered physiological sensor is configured and located to detect a respiratory pattern.

4. The method of claim 3, wherein the respiratory pattern is selected from the group consisting of inspiration, expiration, and respiratory pause.

5. The method of claim 2, wherein the respiratory effort information is a waveform.

6. The method of claim 2, wherein the step of locating includes implanting the self-powered physiological sensor in a subcutaneous, non-vascular location.

7. The method of claim 2, further comprising:
treating sleep disordered breathing by the patient based upon the respiratory effort information.

8. The method of claim 7, further comprising:
implanting a medical device within the patient, including a stimulation element of the medical device located to apply stimulation energy to an upper-airway-patency-related nerve of the patient; and
operating the medical device to deliver stimulation to the upper-airway-patency-related nerve based upon the respiratory effort information.

9. The method of claim 2, further comprising:
monitoring sleep disordered breathing by the patient based upon the respiratory effort information.

10. The method of claim 2, wherein the self-powered physiological sensor is located to sense a small scale bodily movement of a portion of a body of the patient involved in respiration.

11. The method of claim 10, wherein the small scale bodily movement is selected from the group consisting of motion, pressure and strain.

12. The method of claim 10, wherein the small scale bodily movement is selected from the group consisting of an apnea event and a regular respiratory cycle.

13. The method of claim 2, wherein the native tissue is in continuity with pleura of a lung of the patient.

14. The method of claim 2, further comprising:
operating the self-powered physiological sensor to store power generated solely by the self-powered physiological sensor in response to movement of the native tissue.

15. The method of claim 2, further comprising:
conforming the self-powered physiological sensor to the native tissue.

16. The method of claim 15, wherein the self-powered physiological sensor comprises a flexible, resilient material.

17. The method of claim 15, wherein the self-powered physiological sensor comprises a shape memory material.

18. The method of claim 2, further comprising:
operating the self-powered physiological sensor to directly convert mechanical energy to electrical energy.

19. The method of claim 18, wherein the self-powered physiological sensor includes a piezoelectric element.

20. The method of claim 18, wherein the step of operating includes operating the self-powered physiological sensor to electromagnetically convert mechanical energy to electrical energy.

21. The method of claim 18, wherein the step of operating includes operating the self-powered physiological sensor to capacitively convert mechanical energy to electrical energy.
